(19) United States
(12) Patent Application Publication
(10) Pub. No.: US 2004/0022732 A1
(43) Pub. Date: Feb. 5, 2004

(54) Microparticles based on aspartic acid and use thereof as mri contrast agents

(76) Inventors: Rainer Zotz, Wiesbaden (DE); Holger Bengs, Frankfurt am Main (DE)

Correspondence Address: CONNOLLY BOVE LODGE & HUTZ, LLP, 1220 N MARKET STREET, P O BOX 2207, WILMINGTON, DE, 19899

(21) Appl. No.: 10/148,867

(22) PCT Filed: Dec. 4, 2000
(86) PCT No.: PCT/EP00/12156

(51) Int. Cl. A61K 49/00; C07K 14/00

(52) U.S. Cl. 424/9.34; 530/350

(57) ABSTRACT

The present invention relates to microparticles based on aspartic acid with marker substances covalently attached thereto for nuclear magnetic resonance methods, such as the MRI method, to use thereof as contrast agent for these methods, and to a preparation process therefor.

### DESCRIPTION

The present invention relates to microparticles based on aspartic acid with marker substances coupled thereto for nuclear magnetic resonance methods for diagnosis or therapy, such as magnetic resonance imaging, to use thereof for preparing a contrast agent for these methods, and to a preparation process therefor.

Nuclear resonance methods are increasingly being used in medicine for diagnosis and therapy as noninvasive imaging methods which can provide information about pathological changes in the body in a complication-free way without a surgical operation. Demonstration of differences in hydrogen density and relaxation times makes it possible, for example, to identify and localize tumors, edemas, hemorrhages or necrosis in contrast to the healthy surroundings.

The image definition and accuracy are generally increased by administering suitable contrast agents based on paramagnetic substances which, because of their interaction with the surroundings, are able, for example, to influence characteristically the relaxation rate of protons, which makes more accurate evaluation possible.

However, for informative images, it is necessary for the contrast agents to act specifically, i.e. be locally employable, without nonspecific distribution to adjacent tissue and, in addition, show a sufficiently long residence time at the desired site.

Known contrast agents for these methods which are also referred to as MRI (magnetic resonance imaging) methods are Gd(III) compounds such as Gd(III) complex compounds which, because of the large number of seven unpaired electrons of the Gd(III) ion, have a particularly great effect. A compound which is frequently employed as contrast agent is the Gd(III) complex of diethylenetriaminepentaacetic acid (Gd-DTPA) and salts thereof.

However, it has emerged that the paramagnetic substances normally employed as contrast agents show low tissue-specificity. For example, they can be employed to only a very limited extent for investigating the vascular system because they rapidly escape from the bloodstream into the surrounding tissue. In addition, they are degraded by the body within a short time.

DE 42 32 755 describes in general the use of microparticles composed of a copolymer of at least one synthetic polymer and at least one biopolymer as carrier system which can be injected into the blood vessel for active ingredients and diagnostic agents in medicine, in particular for ultrasonic diagnosis. To increase the tissue-specific properties it is proposed in this case to combine the microparticles with substances with appropriate site structure- or tissue-specific properties. Beyond this, the suitability in principle of these microparticles for other diagnostic methods such as the MRI method through linkage of appropriate marker substances with the microparticles is described. There is no reference to the use of aspartic acid and its derivatives.

A study of the biodegradability and the effectiveness as MRI contrast agents of microparticles composed of epichlorohydrin-crosslinked starch and containing attached paramagnetic marker substances is given by “Cross-linked, degradable starch microspheres as carriers of paramagnetic contrast agents for magnetic resonance imaging: Synthesis, degradation, and relaxation properties” P. Rongved et al., Carbohydrate Research, 214 (1991) 325-330. However, it has emerged that particles based on starch circulate in the vascular system for only an inadequate time and are removed from the circulation within seconds.

In view of the rapidly increasing number of vascular disorders, especially in the heart, and the high mortality associated therewith, there was a need for an MRI contrast agent which has high specificity and makes it possible to obtain reliable information about the blood flows specifically in the heart.

It was therefore an object of the present invention to provide a contrast agent for MRI methods which makes it possible specifically to investigate the vascular system and identify pathological divergences of the blood flow, especially in the heart, in order thus to be able to diagnose and treat, at an early time, disturbances of blood flow and the risk of infarction.

This object is achieved by microparticles based on aspartic acid which are characterized in that at least one marker substance for nuclear magnetic resonance methods is covalently attached thereto.

The invention further relates to a contrast agent for MRI methods which contains these microparticles and, where appropriate, a physiological carrier and, where appropriate, further additives and/or excipients in a suitable dosage form.

The present invention relates in particular to the use of the contrast agent for the diagnosis of disorders of the vascular system, in particular of the vascular system of the heart.

According to a further aspect, the invention relates to a process for producing a contrast agent for nuclear resonance magnetic investigation methods using the microparticles of the invention based on aspartic acid with marker substances for such methods covalently bonded thereto.

For the purpose of the invention, “based on aspartic acid” means that the microparticles are composed of polyaspartic acid or a material that is derived from aspartic acid, and may be any derivative thereof, such as, for example, polyaspartic acid-co-imide.

It is essential for use in medicine that the material is well tolerated and non-toxic. The materials are preferably biodegradable.

Microparticles suitable for the invention, materials for producing these microparticles and methods for their production are described, for example, in European patent applications EP 0 458 079 and EP 0 535 387 and in Adv. Mater. 1992; 4; 230-234 “Microparticles from biodegradable Polymers” by Ahlers, M., Krone, V., Waich, A., to which express reference is made herefor for the present invention.

These publications relate specifically to the use of the microparticles described therein as ultrasonic contrast agents.

It has now been found, surprisingly, that these microparticles if produced from materials, derived from aspartic acid are outstandingly suitable as carriers of marker substances for nuclear magnetic resonance methods and are able to increase the residence time of these substances in the vascular system, especially for investigations on the heart.

It is thus possible for the microparticles described therein as ultrasonic contrast agents to be employed directly for the present invention.

It is possible in principle to choose as marker substance any compound which is suitable for nuclear magnetic resonance methods, specifically for medicine, and can be coupled covalently to the microparticles employed according to the invention.

Because of their characteristic effect on proton relaxation, the compounds involved in this case preferably comprise paramagnetic metal ions such as, for example, the elements with atomic numbers 21-29 and 57-70 of the Periodic Table. Examples thereof are iron(II) and iron(III), manganese(II), chromium(III), copper(II), gadolinium(III) and erbium(III), with particular preference for gadolinium(III).

The compounds may be salts or complex compounds such as, for example, chelate complexes.

Ligands frequently employed are the diethylene-triaminepentaacetic acid (DTPA) mentioned above, its salts and derivatives, 1,4,7,10-tetraazacyclodecane-N,N′,N″,N′″-tetraacetate (DOTA), porphyrin systems etc., which are generally known and described comprehensively for this purpose.

The coupling of the marker substances takes place in a manner known per se by, for example, initially linking the ligands to the microparticles and subsequently reacting the particles with the metal ions to give the respective covalently bonded metal complex (see, for example, P. Rongved et al. in: Carbohydrate Research, 214 (1991) 325-330 “Cross-linked, degradable starch microspheres as carriers of paramagnetic contrast agents for magnetic resonance imaging: Synthesis, degradation, and relaxation properties”).

The production of the microparticles of the invention with marker substances coupled thereto as contrast agents for MRI methods, and the dosage of the contrast agent takes place depending on the particular purpose of use in the way customary for the purpose.

The contrast agent produced according to the invention shows a long residence time in the vascular system and does not escape from the vessels into the surroundings. Besides investigation of blood vessels in general, it is particularly suitable also for applications on the heart for investigating the vascular system of the heart, of the myocardium, of the endocardium, for identifying disturbances of blood flow, e.g. for the diagnosis and early identification of infarctions. Very good results can also be achieved in the demonstration of all internal organs and of the brain.

When the microparticles of the invention are used as contrast agents for investigating the blood vessels, the size of the microparticles must be adapted to the vascular system to ensure that the contrast agent can pass satisfactorily through the vessels.

In this case, the size of the microparticles should not exceed 7 μm, and the particle size is preferably in a range from 0.1 μm to 7 μm, in particular 0.1 μm to 3 μm. Thus, it was possible to obtain very good results with particles in a size range from 0.1 μm to 3 μm, in particular with particles of a size of about 2 μm and 3 μm, specifically in the investigation of the heart, especially of the myocardium.

The microparticles used according to the invention can be obtained by, for example, condensing aspartic acid to give polysuccinimide (polyanhydroaspartic acid), opening some of the formed imide rings by nonequivalent addition of, for example, aminoethanol, and esterifying all or some of the introduced hydroxyl groups.

Preference is given to condensation using phosphoric acid and esterification using decanoic acid in a polymer-analogous reaction.

The microparticles which can be employed according to the invention and their production are illustrated in detail below for the example of microparticles composed of polyaspartic acid-co-imide derivatives as obtainable according to EP 0 458 079.

Microparticles composed of polyaspartic acid-co-imide derivatives (PAA-co-imide derivatives) were produced and are, surprisingly, outstandingly suitable as MRI contrast agents. Incorporation of unopened imide rings in particular makes the produced microparticles excellently suspendable in water. In water-containing liquids, the microparticles do not have a sticky, greasy consistency and show scarcely any aggregation. The polymers form a pharmacologically inert matrix under which the marker substances can be coupled. In vivo, these polymers are metabolized to nontoxic, nonallergenic and nonimmunogenic compounds and are excreted.

FIG. 1 shows in a general formula I one embodiment of PAA-co-imide derivatives which can be employed according to the invention.

FIG. 1

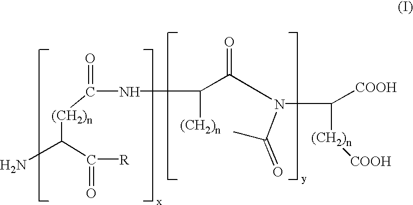

where

n is 1

x is 1 to 500

y is 1 to 500, where

x+y is 2 to 1 000, and

R is O—R1 or NH—R2, in which

R2 is H, (CH₂)ₘ—OR1, (CH₂)ₘ—O—C(O)—R1 or (CH₂)ₘ—O—C(O)—OR1 and

m is 2 to 6, and

R1 is H, aryl, aralkyl, arylalkenyl, alkyl or C3-C8-cycloalkyl or a biologically inactive steroid alcohol or an amino acid, where aryl is unsubstituted or substituted by C1-C4-alkyl, C2-C4-alkenyl, C1-C4-alkylcarbonyloxy, C1-C4-alkoxycarbonyl, C1-C4-alkoxy or hydroxyl,

where the alkyl radicals mentioned for R1 have 1-22 C atoms and the alkenyl radicals have 2-22 C atoms, which are not interrupted or are interrupted by a carbonyloxy or oxycarbonyl group, where the repeating units in square brackets are distributed randomly and/or in blocks in the polymer, and where both the repeating units labeled with x and those labeled with y are identical or different and where the amino acids are α- and/or β-linked, contain.

Aryl means aromatic hydrocarbons such as phenyl and naphthyl, in particular phenyl. In the indicated substituted aryl radicals, preferably 1 to all replaceable hydrogen atoms are replaced by identical or different substituents. The aryl radicals are preferably mono- or disubstituted.

Said alkyl and alkenyl radicals may be both straight-chain and branched.

The biologically inactive steroid alcohols are preferably bonded via their OH group. A preferred steroid alcohol is cholesterol.

The amino acids mentioned for R1 are preferably naturally occurring amino acids such as Tyr, Ala, Ser or Cys, particularly preferably Tyr and Ala. They may be bonded both via their NH2 function and via their COOH function.

The microparticles employed according to the invention may, where appropriate, comprise a gas, consist of or comprise the abovementioned polymers, and be used, mixed with other biocompatible and/or biodegradable polymers or physiologically acceptable excipients, for producing MRI contrast agents for diagnostic or therapeutic methods.

Aspartic acid reacts in a polycondensation reaction to give the corresponding polyimides (polyanhydroaspartic acid, formula II). Partial reaction with one or more compounds of the formulae III and/or IV and/or NH₃

HO—R1 (III)

H₂N—(CH₂)ₘ.OH (IV),

in which m and R1 are as defined above for formula I, results in an α,β-poly-D,L-aspartic ester-co-imide of the formula VIII as depicted below:

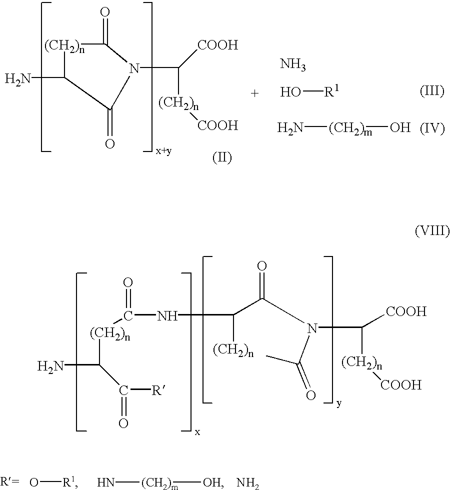

The polyanhydroaminodicarboxylic acid (II) is preferably converted only partly into the open-chain derivative. The proportion of unopened anhydroamino-dicarboxylic acid units in this case is, in particular, 0.1 to 99.9%, preferably 10 to 90% (the percentages relate to a total number of repeating units in the complete polymer). α- or β-linked amino acids are obtained depending on which way the imide ring is opened in the reaction described above.

Compounds of the formulae III and IV which are preferably employed are: 2-aminoethanol, 3-aminopropanol, 2-aminopropanol, alcohols having 1-18 C atoms, in particular methanol, ethanol, isoamyl alcohol and isopropyl alcohol.

A process for preparing α,β-poly-(2-hydroxyethyl)-DL-aspartimide (PHEA) (formula 1: y=0; RNH—CH2-CH2-OH) is described by P. Neri, G. Antoni, F. Benvenuti, F. Cocola, G. Gazzei in J. Med. Chem. vol. 16, 893 (1973). A general method for preparing PHEA is to be found in P. Neri, G. Antoni, Macromol. Synth. vol. 8, 25. Express reference is made to this citation at this point. The reaction takes place in high yield to give a product with a high degree of purity. It is possible in the same way, by substoichiometric use of NH₃ and/or compounds of the formula III and/or IV, to prepare the analogous polyaspartic acid derivative-co-succinimide compounds of the formula VIII (n=1).

The polyaspartamide-co-imides of the formula VIII (R′═HN—(CH₂)ₘ—OH) can then be reacted if necessary in the following reaction step with one or more different, biologically inactive compounds of the formula V and/or VI and/or VII

X—R1 (V)

X—C(0)-R1 (VI)

X—C(0)-OR1 (VII)

to give further polyaminodicarboxylic acid-co-imide derivatives. In this case, X is a leaving group which makes it possible to esterify the polymer alcohol group under mild conditions. Preference is given to chlorine, bromine, iodine, imidazolides, anhydrides or hydroxyl, in particular chlorine.

The reaction with the compounds of the formula type V, VI or VII can take place both with a single such compound and with any combinations of these compounds or else with compounds which have radicals R1 which differ for example in the nature of their branching, in particular differ in their chain length.

The last-mentioned polymer-analogous alkylation or acylation is carried out by known methods of organic chemistry. It proceeds selectively on the hydroxyl function (formula VIII, R′═HN(CH₂)ₘ—OH) to give ethers, esters or carbonates, without attacking other functions on the initial polymer. The Einhorn variant of the Schotten-Baumann acylation in the presence of pyridine is particularly suitable. In this case, very high degrees of derivatization (greater than 70%) are achieved under mild conditions.

The molecular weight of the polymers is generally 200 to 100 000, preferably 3 000 to 70 000. Compounds of the formula type V can be purchased or, if not, can be synthesized in a simple manner by methods known from the literature.

The chloroformic esters (formula VII) are obtained by reacting phosgene with the appropriate biologically inactive, physiologically acceptable, aromatic, araliphatic, aliphatic or cycloaliphatic, in particular unbranched alcohols. The alcohols which are particularly preferably employed are those having an even number of carbon atoms. The chloroformylated steroids are also obtained in this way. It is thus possible in principle to obtain all biologically inactive steroids having reactive hydroxyl groups. Examples which may be mentioned here are: cholesterol, cholestanol, coprostanol, ergosterol, sitosterol or stigmasterol.

The acid chlorides (formula VI) which can likewise be employed are obtained, for example, from the corresponding carboxylic acids by reaction with phosphorus trichloride, phosphorus pentachloride, oxalyl chloride or thionyl chloride.

Compounds of the formula type V, VI or VII in which an alkyl chain is interrupted by an oxycarbonyl or carbonyloxy group are prepared, for example, by reaction of cyclic dicarboxylic anhydrides with alcohols. The dicarboxylic monoesters obtained in this way are then reacted in analogy to the carboxylic acids described above for example with oxalyl chloride to give the corresponding acid chlorides.

The processes described below for producing microparticles can be applied directly to other compounds which can be employed according to the invention for producing the particles as such. One advantageous process for producing the microparticles consists of dissolving one or more of the PAA-co-imide derivatives of the formula II in a solvent or solvent mixture with high melting point or mixing these derivatives with one or more other polymers and/or physiologically acceptable excipients and dissolving in a solvent or solvent mixture with high melting point, and adding dropwise to a condensed cold gas, e.g. liquid nitrogen. Leidenfrost's phenomenon results in absolutely round particles in this case. Examples of solvents which can be employed are alcohols, dimethylformamide, dimethyl sulfoxide, tetrahydrofuran, methylene chloride, dioxane, acetonitrile or mixtures with alcohols. The high-melting and water-miscible solvent is dissolved out for example by transferring the microparticles to water, and the polymer is precipitated at the same time, with the spherical shape of the microparticles being retained.

If the organic solvent used has not only a high melting point but also a low boiling point, this dropwise addition process can be further simplified since the solvent, e.g. tert-butanol, can be removed under mild conditions directly by freeze drying.

Another process for producing the microparticles consists of dissolving one or more of the PAA-co-imide derivatives of the formula I in a solvent or solvent mixture and, where appropriate, after addition of another solvent and/or of one or more other polymers, precipitating or dispersing in water. Examples of other polymers which are suitable are polyvinyl alcohol (Mowiol® 28-99) or polyoxyethylene/polyoxypropylene (®Pluronic F 127). Examples of other solvents which can be used are ethers. Microparticles with a diameter of from 0.5 and up to 15 μm are obtained by vigorous stirring, e.g. with a mixer (25 000 rpm). The solvents are then removed for example by lyophilization.

A particularly advantageous process consists of obtaining the microparticles by spray drying. For this purpose, one or more PAA-co-imide derivatives of the formula I are dissolved, or these derivatives are mixed with one or more other polymers and/or physiologically acceptable excipients and dissolved. Examples of suitable solvents or solvent mixtures are alcohol, dimethylformamide, dimethyl sulfoxide, tetrahydrofuran, methylene chloride, dioxane or acetonitrile. The solution is then sprayed in a spray dryer to give microparticles.

In the described process, the polymers of the formula I can be used alone or else as mixture of various polymers of the formula I. These polymers can also be employed in mixtures with other biodegradable and/or biocompatible polymers (e.g. ®Pluronic F68, PHEA, dextrans, polyethylene glycols, hydroxyethylstarch and other degradable or excretable polysaccharides) or physiologically acceptable excipients (e.g. polymeric plasticizers).

If required, the microparticles may comprise gas, for example air, nitrogen, noble gases such as helium, neon, argon or krypton, hydrogen, carbon dioxide, oxygen, or mixtures thereof. The microparticles are loaded with a gas by, for example, the microparticles being stored after the lyophilization in an appropriate gas atmosphere or being obtained in the spray drying directly on production in an appropriate gas atmosphere.

The MRI contrast agents of the invention are converted before administration by addition of one or more physiologically acceptable carriers and, where appropriate, other additives and/or excipients into a suitable diagnostic or therapeutic dosage form. The physiological isotonicity of the particle suspension can be produced by addition of osmotically active substances, for example sodium chloride, galactose, glucose, fructose.

In the described processes for producing the MRI contrast agents of the invention it is possible to achieve particle sizes with which 90% of the particles are between 0.1 μm and 15 μm. With the spray-drying process it is possible to achieve particle size distributions with which 90% of the particles are smaller than 3 μm. Larger particles are removed by screening out, for example with a 15 μm woven screen and/or 3 μm woven screen. When these microparticles are used as MRI contrast agents for the diagnosis of cardiovascular disorders, particle sizes from 0.1 μm to 7 μm have proved suitable, and particle sizes of from 0.1 μm to 3 μm are advantageously employed.

The invention is illustrated in more detail below by means of some selected examples.

### EXAMPLE 1

### Preparation of polysuccinimide-co-α,β-(hydroxyethyl)-D,L-aspartamide (70:30)

10 g (103 mmol) of polyanhydroaspartic acid (are dissolved in about 40 ml of N,N-dimethylformamide (DMF) where appropriate with cautious heating. 1.83 g (30 mmol) of freshly distilled 2-aminoethanol are added dropwise to this solution, and the mixture is stirred at room temperature overnight. The reaction mixture is precipitated in butanol and washed several times with dry acetone. The drying takes place in vacuo at elevated temperature. The yield of white, water-soluble product is almost 100% and it is checked by NMR spectroscopy for DMF and butanol residues. The molar ratio of polyanhydroaspartic acid to aminoethanol employed corresponds approximately to the copolymer composition.

### EXAMPLE 2

### Preparation of n-butyl 4-chloro-4-oxobutyrate

Monobutyl succinate is mixed with excess thionyl chloride and a drop of DMF. The reaction takes place with evolution of gas. The mixture is left to stir with exclusion of moisture overnight, and then the excess thionyl chloride is distilled off under atmospheric pressure. The remaining crude product is fractionally distilled under 0.05 mbar, and the pure product is obtained at about 70° C. In a characterization by IR spectroscopy, the product shows bands at 1 800 cm⁻¹ (acid chloride) and 1 740 cm⁻¹ (ester) of equal intensity.

### EXAMPLE 3

### Preparation of polysuccinimide-co-α,β-(butyloxycarbonyl-propionyloxyethyl)-D,L-aspartamide (70:30)

6 g of polysuccinimide-co-α,β-(hydroxyethyl)-D,L-aspartamide (=16 mmol of hydroxyethyl groups), prepared as described in example 1, dissolved in 100 ml of dry N,N-dimethylformamide (DMF). Addition of 4 g (50 mmol) of pyridine is followed by cooling to 0° C. and addition while stirring over the course of 15 minutes of 4.8 g (25 mmol) of n-butyl 4-chloro-4-oxobutyrate (see example 2). The mixture is stirred overnight and precipitated in 0.5 l of ether. The precipitated product is filtered off with suction and washed with ether, acetone, water, acetone and ether. About 8 g of a white polymer with a degree of substitution of approximately 100% (can be checked by NMR spectroscopy) are obtained. The resulting polymer is soluble for example in acetonitrile with a trace of dimethyl sulfoxide (DMSO), in DMSO or DMF.

### EXAMPLE 4

### Preparation of polysuccinimide-co-α,β-(nonylcarbonyl-oxyethyl)-D,L-aspartamide (50:50)

6 g of a polysuccinimide-co-α,β-(hydroxyethyl)-D,L-aspartamide (50:50)\hat{=}(24 mmol of hydroxyethyl groups), each was prepared in analogy to example 1 from poly-anhydroaspartic acid (MW=14 000) and 2-aminoethanol (molar ratio 2:1), are dissolved in 100 ml of dry DMF and, after addition of 8 g (100 mmol) of dry pyridine, cooled to 0° C. 9.6 g of distilled decanoyl chloride are slowly added dropwise, and further processing is in analogy to example 3. About 8 g of a white, completely substituted polymer (NMR check) are obtained, the polymer being soluble for example in dichloromethane and THF with in each case a trace of DMSO or in methanol/dichloromethane mixtures.

### EXAMPLE 5

### Preparation of polysuccinimide-co-α,β-(nonylcarbonyl-oxyethyl)-D,L-aspartamide Varying in copolymer Composition and Varying in Molecular Weight

Various polysuccinimide-co-α,β-(hydroxyethyl)-0,L-aspartamides inter alia of the composition 70:30, 50:50 and 30:70 were prepared from polyanhydroaspartic acids varying in molecular weight (MW=7 000; about 13 000; 30 000) in analogy to example 1 and reacted with decanoyl chloride as described in example 4 to give the corresponding polysuccinimide-co-α,β-(nonylcarbonyloxy-ethyl)-D,L-aspartamides.

a)—Polysuccinimide-co-α,β-(nonylcarbonyloxyethyl)-D,L-aspartamide (70:30) from polyanhydroaspartic acid (MW=7 000); characterized by NMR

b)—Polysuccinimide-co-α,β-(nonylcarbonyloxyethyl)-D,L-aspartamide (70:30) from polyanhydroaspartic acid (MW=14 000); characterized by NMR

c)—Polysuccinimide-co-α,β-(nonylcarbonyloxyethyl)-D,L-aspartamide (70:30) from polyanhydroaspartic acid (MW=30 000); characterized by NMR

d)—Polysuccinimide-co-α,β-(nonylcarbonyloxyethyl)-D,L-aspartamide (30:70) from polyanhydroaspartic acid (MW=12 000); characterized by NMR

### EXAMPLE 6

### Preparation of polysuccinimide-co-α,β-(octyloxy-carbonyloxyethyl)-D,L-aspartamide (70:30)

6 g of polysuccinimide-co-α,β-(hydroxyethyl)-0,L-aspartamide (70:30) (\hat{=}16 mmol of hydroxyethyl groups), prepared as described in example 1 from polyanhydroaspartic acid (MW=37 000) and aminoethanol, are reacted in analogy to example 3 with 4.8 g (25 mmol) of octyl chloroformate and worked up correspondingly. About 8 g of a white, completely substituted polymer, which is soluble in THF or methanol/dichloromethane mixtures, are obtained.

### EXAMPLE 7

### Preparation of polysuccinimide-co-α,β-(nonylcarbonyl-oxyethyl)-co-α,β-(hydroxyethyl)-D,L-aspartamide (60:20:20)

6 g of polysuccinimide-co-α,β-(hydroxyethyl)-D,L-aspartamide (60:40) (>>66 0 mmol of hydroxyethyl groups), which was prepared in analogy to example 1 from polyanhydroaspartic acid and 2-aminoethanol (molar ratio 6:4), are reacted in analogy to example 3 with 2.3 g of decanoyl chloride (\hat{=}12 mmol). Because conversion was incomplete (relatively small excess of acid chloride), the free OH groups are only half esterified. About 7 g of a white polymer are produced. Microparticles of this substance show a solid consistency in water and are readily suspendable.

### EXAMPLE 8

### Preparation of polysuccinimide-co-α,β-(oleyloxyethyl)-D,L-aspartamide (10:90)

6 g of polysuccinimide-co-α,β-(hydroxyethyl)-D,L-aspartamide (10:90) (\hat{=}40 mmol of hydroxyethyl groups), prepared in analogy to example 1 with a molar ratio of polyanhydroaspartic acid to 2-aminoethanol of 1:9, are reacted with 20 g of distilled oleyl chloride in analogy to example 3. The heterogeneous reaction mixture becomes homogeneous on addition of dichloro-methane. It is precipitated twice in methanol which is cooled to −20° C. The yellowish-colored polymer is thermoplastic.

### EXAMPLE 9

### Production of Microparticles

40 mg of polysuccinimide-co-α,β-(nonylcarbonyloxy-ethyl)-D,L-aspartamide (50:50) from example 4 are dissolved in 1 ml of methylene chloride/methanol (50/1 part by volume). The solution is introduced with stirring (800 rpm) into a beaker containing 60 ml of 0.1% by weight aqueous polyvinyl alcohol solution (®Mowiol 28-99) which is saturated with 0.3 ml of methylene chloride/methanol (50/1). At the same time, the solution is finely dispersed with a mixer (25 000 rpm).

After 5 minutes, the contents are put in a beaker containing 200 ml of water and stirred (200 rpm) for 30 minutes. The supernatant water is decanted off, and the microparticles are lyophilized (diameter after lyophilization: 0.5 to 15 um).

### EXAMPLE 10

### Production of Microparticles

80 mg of polysuccinimide-co-α,β-(octylcarbonyloxy-ethyl)-D,L-aspartamide (70:30) from example 6 are dissolved in 1 ml of dimethyl sulfoxide at 50° C. and 20 mg of hydroxypropylcellulose (®Klucel M.) are added. The solution of the two polymers is added dropwise using a cannula (disposable syringe, cannula external diameter 0.6 mm) to liquid nitrogen (100 ml).

The resulting microparticles are transferred into 200 ml of water and extracted from the remaining solvent for 2 hours. Excess water is decanted off, and the microparticles are lyophilized (diameter after lyophilization: 1-2 um).

### EXAMPLE 11

### Production of Microparticles

4 g each of polysuccinimide-co-α,β-(octyloxycarbonyl-oxyethyl)-D,L-aspartamide (A) (example 6) and polysuccinimide-co-ci,β-(nonylcarborlyloxyethyl)-D,L-aspartamide (B) (example 5d) are dissolved at a concentration of 2% in the solvents indicated in table 1. The polymers are then sprayed in a spray dryer (Mini Spray Dryer Büchi 190, Büchi, W. Germany) to give microparticles.

The size distribution of the microparticles was determined in a Cilas 715 granulometer.

30 mg portions of each of the microparticles produced above are dispersed in 1.5 ml of suspending aid. The suspending aids consist of 150 mg of dextran 40 (Roth, W. Germany), 7.5 mg of polysorbate and 13.5 mg of NaCl in 1.5 ml of distilled water. The suspensions are filtered using woven screens (15 um and 3 μm mesh widths) and then lyophilized. Before administration, the microparticles are suspended in water.

| | | Particle size | | |
|---|---|---|---|---|
| | | 10% | 50% | 90% |
| | | smaller | smaller | smaller |
| Substance | Solvent | than | than | than |
| A | THF | 1.6 μm | 3.6 μm | 6.7 μm |
| B | CH₂Cl₂ | 2.4 μm | 3.6 μm | 6.7 μm |
| B | CH₂Cl₂/methanol | 1.3 μm | 1.9 | 3.0 μm |
| | 3.6:1 (vol.) | | | |
| B | CH₂Cl₂/methanol | 1.2 μm | 1.8 μm | 2.6 μm |
| | 2:3 (vol.) | | | |
| B | THF/methanol | 1.3 μm | 1.9 μm | 2.7 μm |
| | 3.6:1 (vol.) | | | |

1. Microparticles based on aspartic acid, characterized in that at least one marker substance for nuclear magnetic methods is covalently attached thereto.

2. Microparticles as claimed in claim 1, characterized in that the aspartic acid material is selected from one or more polyaspartic acid-co-imide.

3. Microparticles as claimed in claim 1 or 2, characterized in that a compound of a paramagnetic metal ion is selected as marker substance.

4. Microparticles as claimed in any of the preceding claims, characterized in that the paramagnetic metal ion is gadolinium(III).

5. Microparticles as claimed in any of the preceding claims, characterized in that the marker substance is selected from a complex compound of the metal ion with diethylenetriaminepentaacetic acid, a salt thereof, 1,4,7,10-tetraazacyclodecane-N,N′,N″,N′″-tetraacetate and a phorphyrin system.

6. Microparticles as claimed in any of the preceding claims, characterized in that the polyasparagine material employed is a polyasparagine-co-imide derivative of the following formula I 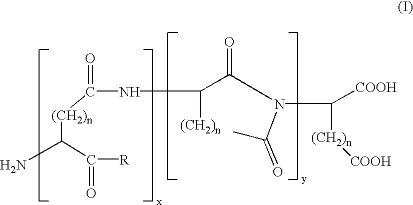
where
n is 1
x is 1 to 500
y is 1 to 500, where
x+y is 2 to 1 000, and
R is O—R1 or NH—R2, in which
R2 is H, (CH₂)ₘ—OR1, (CH₂)ₘ—O—C(0)-R1 or (CH₂)ₘ—O—C(0)-OR1 and
m is 2 to 6, and
R1 is H, aryl, aralkyl, arylalkenyl, alkyl or C3-C8-cycloalkyl or a biologically inactive steroid alcohol or an amino acid, where aryl is unsubstituted or substituted by C1-C4-alkyl, C2-C4-alkenyl, C1-C4-alkylcarbonyloxy, C1-C4-alkoxycarbonyl, C1-C4-alkoxy or hydroxyl,
where the alkyl radicals mentioned for R1 have 1-22 C atoms and the alkenyl radicals have 2-22 C atoms,
which are not interrupted or are interrupted by a carbonyloxy or oxycarbonyl group, where the repeating units in square brackets are distributed randomly and/or in blocks in the polymer, and where both the repeating units labeled with x and those labeled with y are identical or different and where the amino acids are α- and/or β-linked, contain.

7. Microparticles as claimed in claim 6, characterized in that in formula I
R is NH—R2
m is 2 and
R1 is H, aryl, aralkyl, alkyl or C5-C6-cycloalkyl, where the alkyl radicals have 1-22 C atoms.

8. Microparticles as claimed in claim 6, characterized in that in formula I
R is O—R1 and
R1 is H, aryl, aralkyl, alkyl or C5-C6-cycloalkyl where the alkyl radicals have 1-22 C atoms.

9. A contrast agent for MRI methods comprising microparticles as claimed in any of claims 1 to 8 and, where appropriate, a physiological carrier and/or other additives and/or aids in a suitable dosage form.

10. The use of contrast agents as claimed in claim 9 for producing diagnostic or therapeutic compositions.

11. The use of contrast agents as claimed in claim 9 for the diagnosis of disorders of the blood vessels.

12. The use of contrast agents as claimed in claim 9 for the diagnosis of disorders of the heart.
